(19) United States
(12) Patent Application Publication
(10) Pub. No.: US 2021/0260234 A1
(43) Pub. Date: Aug. 26, 2021

(54) ANTIMICROBIAL DEVICE

(71) Applicants: Robert L. Esse, Monticello, MN (US); Claire M. Troxel, Monticello, MN (US); David Egberg, Bonita Springs, FL (US)

(72) Inventors: Robert L. Esse, Monticello, MN (US); Claire M. Troxel, Monticello, MN (US); David Egberg, Bonita Springs, FL (US)

(21) Appl. No.: 17/196,955

(22) Filed: Mar. 9, 2021

Related U.S. Application Data
(62) Division of application No. 15/876,765, filed on Jan. 22, 2018, now abandoned.
(60) Provisional application No. 62/550,356, filed on Aug. 25, 2017.

(51) Int. Cl. A61L 2/238; A61L 2/00

(52) U.S. Cl. CPC A61L 2/238; A61H 3/02; A61L 2/0082

(57) ABSTRACT

A device for reducing contaminants on a user's hands, the device having an antimicrobial surface with at least 60% copper. The device may have a rounded shape with a length of between approximately 2 inches and 5 inches, and a width between approximately 1 inch and 4 inches, and a depth between approximately 0.25 inches and 1 inch. In some embodiments, the antimicrobial surface may be an entire outer surface of the device. In some embodiments, the antimicrobial surface may include at least 70%, at least 80%, or at least 90% copper. In other embodiments, the antimicrobial surface may include 100% copper. Moreover, in some embodiments, the antimicrobial surface may have at least one of tin, nickel, aluminum, and zinc.

### CROSS-REFERENCE TO RELATED APPLICATIONS

The present disclosure claims priority to Provisional Application No. 62/550,356, entitled Antimicrobial Device, and filed Aug. 25, 2017, the content of which is hereby incorporated by reference herein in its entirety.

### FIELD OF THE INVENTION

The present disclosure relates to novel and advantageous devices for reducing microbiological contaminants. Particularly, the present disclosure relates to novel and advantageous devices for reducing microbiological contaminants on skin. More particularly, the present disclosure relates to novel and advantageous handheld and copper-based devices for reducing microbiological contaminants on a user's hands.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Human interface with others is a social exercise that often includes the shaking of hands, hugging, touching, etc., which may promote the exchange of bacteria and other contaminants on hands. Door handles, such as restroom door handles and other door handles may additionally add to the bacteria on hands, as door handles, and in particular public door handles, are commonly touched by several people in a relatively short period of time. These encounters may bring forth contacts that involve the transfer of unwanted and living bacteria on hands, which can be transferred internally through an individual's casual touching of their face, nose, or mouth, or during the consumption of food.

While hand washing with soap and water may reduce or eliminate hand bacteria, this can be inconvenient in many settings. Moreover, and particularly with respect to public restrooms, an individual's hands may be re-contaminated upon contact with faucet fixtures, door handles, or other surfaces before exiting the restroom. Liquid sanitizing solutions such as ethyl alcohol-based solutions may allow an individual to sanitize hand bacteria in between washings, but sanitizing solutions can be messy and drying. Moreover, to be effective, sanitizing solutions often need time to air dry on a user's hands. Sanitizing solutions can also spill or leak.

Thus, there is a need in the art for devices for cleaning or sanitizing a user's hands on the go or in between washings. In particular, there is a need in the art for devices for destroying or minimizing bacteria on a user's hands without the use of water or other liquids. More particularly, there is a need in the art for relatively small, handheld devices for destroying or minimizing bacteria on a user's hands without the use of water or other liquids.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a device for reducing contaminants on a user's hands, the device having an antimicrobial surface with at least 60% copper. The device may have a rounded shape with a length of between approximately 2 inches and 5 inches, and a width between approximately 1 inch and 4 inches, and a depth between approximately 0.25 inches and 1 inch. In some embodiments, the antimicrobial surface may be an entire outer surface of the device. In some embodiments, the antimicrobial surface may include at least 70%, at least 80%, or at least 90% copper. In other embodiments, the antimicrobial surface may include 100% copper. Moreover, in some embodiments, the antimicrobial surface may have at least one of tin, nickel, aluminum, and zinc.

The present disclosure, in one or more embodiments, additionally relates to a method of manufacturing an antimicrobial device for reducing contaminants on a user's hands. The method may include delineating first and second portions of the device from a metal plate comprising at least 60% copper, shaping the first and second portions to be convex, and coupling the first and second convex portions together to form a hollow rounded shape. In some embodiments, coupling the first and second convex portions together may include at least one of soldering, welding, and brazing the portions together. In some embodiments, the first and second portions may each have a joining edge having a toothed surface configured to engage with a joining edge of the opposing portion. In some embodiments, the first portion may have a joining edge with a lip, and the second portion may have a joining edge with a groove configured to receive the lip of the first portion. Moreover, in some embodiments, the method may include treating the device with an acid wash, alkali wash, photochemical treatment, or physical abrasion.

The present disclosure, in one or more embodiments, additionally relates to a method of reducing contaminants on an individual's hands, the method including weaving a plurality of strands into a fabric, each strand including at least 60% copper, and applying the fabric to a handle of an object. The object may be a crutch, walker, or bicycle in some embodiments. Moreover, the method may include treating the fabric with an acid wash, alkali wash, photochemical treatment, or physical abrasion.

The present disclosure, in one or more embodiments, additionally relates to an antimicrobial device for reducing contaminants on a user's hands, the device having an antimicrobial surface with at least 60% copper, and the device additionally configured for a second function. In some embodiments, the device may be a keychain, zipper pull, or smartphone case.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1A is a top view of a handheld antimicrobial device of the present disclosure, according to one or more embodiments.

FIG. 1B is a side view of the handheld antimicrobial device of FIG. 1A, according to one or more embodiments.

FIG. 2 is a side view of another handheld antimicrobial device of the present disclosure, according to one or more embodiments.

FIG. 3A is a top view of another handheld antimicrobial device of the present disclosure, according to one or more embodiments.

FIG. 3B is a side view of the handheld antimicrobial device of FIG. 3A, according to one or more embodiments.

FIG. 4 is a side view of a keychain antimicrobial device of the present disclosure, according to one or more embodiments.

FIG. 5 is a side view of a zipper pull antimicrobial device of the present disclosure, according to one or more embodiments.

FIG. 6 is a side view of a cellphone case antimicrobial device of the present disclosure, according to one or more embodiments.

FIG. 7 is a top view of two halves of an antimicrobial device of the present disclosure, according to one or more embodiments.

FIG. 8A is a top view of a handheld antimicrobial device of the present disclosure, according to one or more embodiments.

FIG. 8B is a side view of the antimicrobial device of FIG. 8A, according to one or more embodiments.

FIG. 9A is a side view of an antimicrobial fabric of the present disclosure, according to one or more embodiments.

FIG. 9B is a side view of a crutch having an antimicrobial fabric arranged on a handle thereof, according to one or more embodiments.

FIG. 9C is a perspective view of a walker having an antimicrobial fabric arranged on handles thereof, according to one or more embodiments.

FIG. 9D is a perspective view of a bicycle having an antimicrobial fabric arranged on handles thereof, according to one or more embodiments.

FIG. 9E is a perspective view of a sheath of antimicrobial fabric of the present disclosure, according to one or more embodiments.

FIG. 10A is a photograph of a petri dish in which a copper chip was overlaid with a seeded agar, before incubation.

FIG. 10B is a photograph of a petri dish in which a copper chip was arranged over a seeded agar, before incubation.

FIG. 10C is a photograph of a petri dish in which a seeded agar was arranged, before incubation.

FIG. 11A is a photograph of a petri dish in which a copper chip was overlaid with a seeded agar, after incubation.

FIG. 11B is a photograph of a petri dish in which a copper chip was arranged over a seeded agar, after incubation.

FIG. 11C is a photograph of a petri dish in which a seeded agar was arranged, after incubation.

### DETAILED DESCRIPTION

The present disclosure relates to novel and advantageous devices for reducing or eliminating bacteria or other contaminants on a user's hands without the use of water or other liquids. In particular, the present disclosure relates to handheld and copper-based products that may be rubbed on or between a user's hands to reduce or eliminate contaminants on the user's hands relatively discreetly and without the need for water or other liquids.

Recently, it has been recognized that surfaces containing varying levels of copper, bronze, and brass may have antimicrobial properties that may kill bacteria, mold, and viruses when in physical contact. Moreover, tests have been reported showing efficacy against S. aureus, E. aerogenes, MRSA, P. aeruglinosa, and E. coli O157:H7. See “Antimicrobial Copper,” Copper Development Association, https://www.antimicrobialcopper.org/us/epa-registration. It is believed that the antimicrobial mechanism of copper and copper alloys is multi-faceted. Copper is generally oxidized at its surface, forming cuprous ion and superoxide. When a microbe is in contact with a copper surface, these cuprous ions may operate to destroy the microbe by a number of mechanisms, including but not limited to the following: lipid peroxidation, enzyme inhibition, surface protein destruction, respiratory chain inhibition, and cell membrane destruction. See also Espirito Santo C, “Bacterial Killing by Dry Metallic Copper Surfaces,” Appl. Environ. Microbiol. 2011 February; 77(3):794-802; Wilks, S A, “The survival of Escherichia coli O157 on a Range of Metal Surfaces,” International Journal of Food Microbiology, 105(3): 445-54; Michels, H. T., “Copper Alloys for Human Infectious Disease Control,” Presented at Materials Science and Technology Conference, Sep. 25-28, 2005, Pittsburgh, Pa., Copper for the 21st Century Symposium.

In the U.S., to qualify copper and its alloys as registered antimicrobial substances under federal pesticide regulations, testing under Good Laboratory Practice guidelines by an EPA-approved laboratory was required by the EPA. After these tests were concluded in 2008, registrations of various copper alloys were granted. See EPA Registration Nos. 82012-1, 82012-2, 82012-3, 82012-4, 82012-5, and 82012-6. All of the registered alloys have minimum nominal copper concentrations of 62%. Moreover, the EPA labeling guidelines indicated that normal tarnishing did not impair antimicrobial effectiveness.

In some embodiments, an antimicrobial device of the present disclosure may be constructed of, or include, an effective amount copper or another antimicrobial substance. In some embodiments, the device may have a quantity of copper, by weight, of between approximately 60% and approximately 100%. For example, in some embodiments, the device may be formed of, or may have one or more surfaces formed of, pure copper. In other embodiments, the device may be formed with, or may have one or more surfaces formed with, approximately 60%, 65%, 70%, 75%, 80%, 85%, 90%, or 95% copper. In still other embodiments, the device may have, or may have one or more surfaces with, any other suitable percentage by weight of copper. In general, a higher percentage of copper, or another antimicrobial substance, may increase the antimicrobial effectiveness of the device. That is, a device formed of 100% copper may be more effective in minimizing bacteria and other contaminants than a device formed with 60% copper. In some embodiments, an antimicrobial device of the present disclosure may be provided with different effectiveness ratings. In some embodiments, the device may be formed with, or may have one or more surfaces formed with, one or more copper alloys. Suitable metal alloys may include, but are not limited to, such materials as copper tin, copper zinc or copper nickel. Multiple copper alloys may be used in some embodiments. Additionally, in some embodiments, the device may contain other metals, metal alloys, and/or other suitable materials.

In some embodiments, a device of the present disclosure may include one or more additives, such as tin, zinc, lead, phosphorous, aluminum, manganese, and/or other additives. These and/or other additives may provide attributes such as increased hardness, without reducing, or without significantly reducing, the antimicrobial effectiveness of the device. Still other metals or materials may be added to produce desired properties for a given application. In some embodiments, an antimicrobial device of the present disclosure may be constructed of, or include a quantity of, bronze. Bronze often includes a combination of approximately 90% copper with approximately 10% tin and/or other metals, such as phosphorous, aluminum, or manganese. In some embodiments, an antimicrobial device of the present disclosure may be constructed of, or include a quantity of, brass. Brass often includes a combination of approximately 90% copper to approximately 10% zinc and/or other materials.

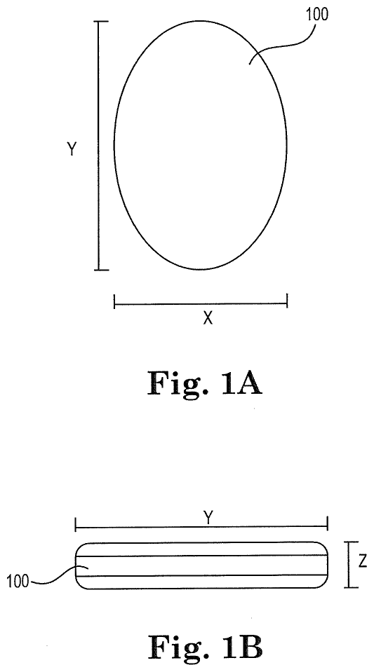

FIGS. 1A and 1B show one example of a handheld antimicrobial device 100 of the present disclosure, according to one or more embodiments. The device 100 may have any suitable dimensions, and in some embodiments may be sized and/or shaped to fit within the palm of a user's hand. For example, the device 100 may have a first dimension, such as a width x, a second dimension, such as a length y, and a third dimension, such as a depth z. In some embodiments, each of the width x, length y, and depth z may be between approximately 0.25 inches and approximately 6 inches. Moreover, in some embodiments, the device 100 may be provided with different sizes to fit in the palm of different users' hands. For example, the device 100 may be provided and configured for a children's size and adults' size. Additionally, in some embodiments, the device 100 may be provided and configured for a women's size and men's size. In other embodiments, the device 100 may be provided with small, medium, and large sizes. In still other embodiments, the device 100 may be provide with other suitable sizing.

In one or more embodiments, a children's sized device 100 may have a width x of between approximately 0.75 inches and approximately 1.75 inches, a length y of between approximately 1.25 inches and approximately 2.5 inches, and a depth z of between approximately 0.25 inches and approximately 1 inch, or a depth z of approximately 0.5 inches.

Additionally, in one or more embodiments, a women's sized device 100 may have a width x of between approximately 1.5 inches and approximately 2.5 inches, a length y of between approximately 1.75 inches and approximately 3 inches, and a depth of between approximately 0.25 inches and approximately 1 inch, or a depth z of approximately 0.5 inches.

Additionally, in one or more embodiments, a men's sized device 100 may have a width x of between approximately 1.75 inches and approximately 3 inches, a length y of between approximately 2.5 and approximately 5 inches, and a depth z of between approximately 0.5 and approximately 2 inches, or a depth z of approximately 0.5 inches.

In other embodiments, the device 100 may have any other suitable dimensions, and may have one or more dimensions smaller or larger than those described above. The device 100 may generally be solid or hollow, and thus the walls of the device may have any suitable thickness. In general, the walls of the device 100 may have a thickness configured to prevent them from easily bending or deforming from use.

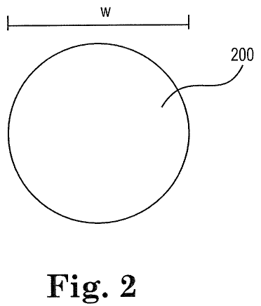

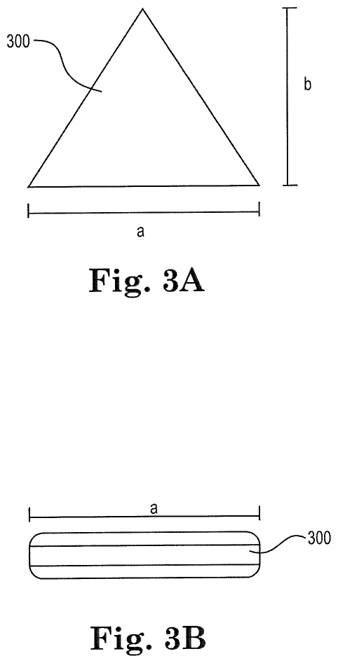

The device 100 may generally be shaped such that it may be easily held in a user's palm and easily rubbed between a user's hands. For example, the device 100 may have one or more smooth sides, such that the device may generally feel relatively smooth to a user's touch. Where the device 100 has corners and/or edges, the corners and/or edges may be rounded in some embodiments. In this way, the device 100 to be easily stored in a pocket, purse, or backpack, for example, without catching on any corners. As shown in FIG. 1A, the device 100 may have a generally oval or egg-like shape in some embodiments, with rounded sides. As shown in FIG. 2, in some embodiments, the device 200 may have a spherical shape with a diameter w of between approximately 1 inch and approximately 3 inches. As shown in FIGS. 3A and 3B, in some embodiments, the device 300 may have a triangular shape with a width a, length b, and a depth c. The width a and length b may each be between approximately 1 and approximately 6 inches. The depth c may be between approximately 0.25 and approximately 2 inches. In other embodiments, the devices 200, 300 may have smaller or larger dimensions. Moreover, as described above, the devices 200, 300 may be provided with different sizes, such as a children's size, women's size, and men's size. In still other embodiments, an antimicrobial device of the present disclosure may have any other suitable shape and/or dimensions.

In some embodiments, an antimicrobial device of the present disclosure may have one or more ergonomic elements. For example, an antimicrobial device of the present disclosure may have one or more grooves or contours configured to receive a user's thumb or finger while the user holds the device. The device may have one or more grooves or contours configured for receiving a user's palm.

Additionally or alternatively, in some embodiments, an antimicrobial device of the present disclosure may have one or more gripping, and/or massage elements. For example, an antimicrobial device may have one or more rubber or rubberized surfaces. In some embodiments, an antimicrobial device may have rubber, plastic, or other material protuberances, nubs, nobs, protrusions, ridges, ribs, or other components on one or more surfaces. Such protuberances or other components may provide increased grip as a user holds or rubs the device. Additionally, such protuberances or other components may provide a massaging or soothing effect on a user's hands.

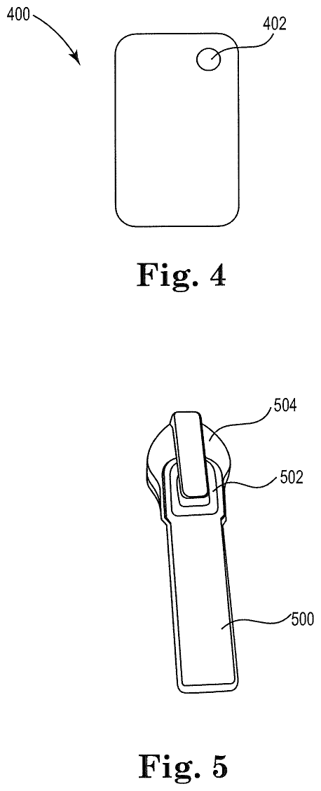

In some embodiments, an antimicrobial device of the present disclosure may be configured to be generally mobile, such that a user may be able to carry the device in a pocket, in a purse or backpack, or in another relatively easily accessible and/or mobile location. For example, the device 100 shown in FIGS. 1A and 1B may be generally carried in a user's pocket, purse, backpack, or otherwise on the user's person. However, other devices are contemplated as well. For example, the device may generally be a functional device that may be usable for purposes in addition to reducing or eliminating contaminants. As shown for example in FIG. 4, a device 400 of the present disclosure may be configured as, or may operably function as, a keychain. The device 400 may have any suitable or desirable shape. While the device 400 in FIG. 4 is shown with a rectangular shape, in other embodiments, the device may have any other suitable or desirable shape. For example, the device 400 may have a round, triangular, or other geometric shape, or may be shaped as an animal, character, or generally any other known shape. As shown in FIG. 4, the device 400 may have an opening 402 configured to receive a keyring.

FIG. 5 shows another embodiment of a device 500 of the present disclosure which may have additional functionality for another purpose. As shown, the device 500 may be configured to provide a pull-tab for a zipper. The zipper pull 500 may have an opening or loop 502 configured for coupling to a slider 504 of a zipper. The zipper pull 500 may generally have a rectangular shape configured to extend outward from the slider 504 of the zipper. The zipper pull 500 may be sized and shaped to be grasped with a thumb and forefinger, such that a user may operate the attached zipper slider 504. In this way, the device 500 may be configured to provide antimicrobial benefits each time a user uses the zipper mechanism. The zipper may be arranged on, for example, a jacket, coat, purse, backpack, or other article or accessory.

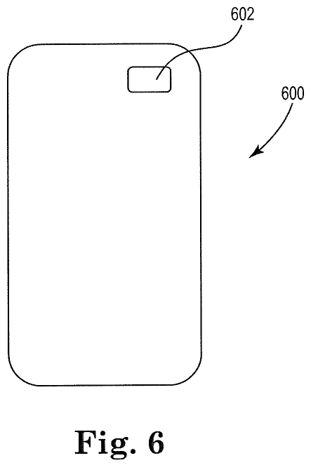

FIG. 6 shows another embodiment of a device 600 of the present disclosure that may have additional functionality. As shown in FIG. 6, the device 600 may be, or may be configured for use as, a cellphone case or smartphone case. The device 600 may generally have a rectangular shape with rounded or squared corners to accommodate a smartphone. The device 600 may additionally define a recess on one side with a hollow depth and configured to receive the smartphone. The device 600 may have an opening 602 through which a smartphone camera may be exposed when the phone is arranged within the case. In some embodiments, the device 600 may have padding, a liner, or one or more inserts to facilitate a friction fit or other fit between the smartphone and the device. In some embodiments, an entire outer portion or outer surface of the smartphone case 600 may be constructed of, or may contain, a quantity of copper. However, in other embodiments, a smaller portion of the case 600 may be constructed with, or contain, a quantity of copper. For example, the smartphone case 600 may have a copper insert arranged on an outer surface of the case. As a user holds or carries his or her smartphone in his or her hand, such as to talk, text, email, take photos, or use applications, the smartphone case 600 may provide antimicrobial effects by contacting the user's hands. Similarly, a device of the present disclosure may be a tablet or e-reader case, or a laptop case.

In some embodiments, a device of the present disclosure, or a portion thereof, may be treated with a surface treatment configured to aid in oxidation of copper and/or increase ion density. Such treatments may improve the efficacy of the antimicrobial device in some embodiments. In particular, where copper ions are at least partially responsible for the antimicrobial action of the device, cleaning the device may enhance performance by increase a number of copper ions on the surface of the device. For example, in some embodiments, the device may be treated with an acid wash. An acid wash may include washing, scrubbing, soaking, rinsing, or otherwise treating a surface of the device with a bleach, chlorine bleach, or hydrogen peroxide solution. An acid wash may oxidize the copper. In other embodiments, the device may be treated with an alkali wash. An alkali wash may include washing, scrubbing, soaking, rinsing, or otherwise treating a surface of the device with a solution of one or more bases, such as sodium hydroxide or potassium hydroxide. In other embodiments, the device may be treated with a photochemical treatment or abrasion. In some embodiments, one or more acid wash, alkali wash, photochemical, abrasion, or other treatments may be applied to an antimicrobial device before a first use, for example. Such washes or treatments may remove or reduce oils, lanolin, and other debris from the surface of the device and increase the number of copper ions on the surface. In some embodiments, a device of the present disclosure may be treated or washed prior to first use. For example, in some embodiments, a device of the present disclosure may be provided to a user with instructions to wash the device in hydrogen peroxide or chlorine bleach prior to using the device. In some embodiments, the device may be provided to a user with an initial coating or oil, such that the coating or oil may be removed by the initial washing of the device. Additionally or alternatively, one or more treatments may be applied to an antimicrobial device between uses.

As described above, an antimicrobial device of the present disclosure may comprise one or more metals in some embodiments. For example, an antimicrobial device may include copper, bronze, or brass, together with any suitable additives. In some embodiments, the metal(s) and/or additives may be combined by melting. That is, the one or more metals may be melted together, and the one or more additives may be added to the molten mixture. In some embodiments, the molten mixture may be poured into one or more molds for forming or casting an antimicrobial device of the present disclosure. In other embodiments, hammering, rolling, or other metal forming methods may be used to shape and form an antimicrobial device of the present disclosure. In still other embodiments, suitably sized and shaped pieces may be stamped, cut, punched, or otherwise delineated from a sheet or plate of material. The suitably sized and shaped pieces may be joined together using soldering, brazing, welding, an adhesive material, or other suitable joining methods to create an antimicrobial device.

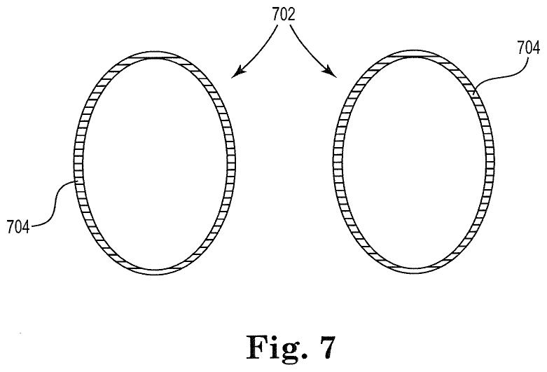

As a particular example, to form the antimicrobial device 100 shown in FIGS. 1A and 1B, two flattened ovals may be stamped, cut, punched, or otherwise delineated from a sheet or plate of material. Each oval may be configured to form approximately half of the device 100. Each oval may have a length y and a width x similar to approximately that of the device 100. Each of the two ovals may be pressed, hammered, or otherwise manipulated to have a convex or concave shape with a hollow depth, as shown in FIG. 7. For example, the hollow depth of each piece may be approximately half the depth of the device 100 of FIGS. 1A and 1B, or approximately (½)z. The two convex or concave oval pieces 702 may be configured to be coupled together, to form the egg-shaped device 100 shown in FIGS. 1A and 1B. In some embodiments, each convex or concave oval piece 702 may have a joining edge 704. The joining edge 704 may surround a perimeter of the concave or convex oval piece 702. For example, the joining edge 704 may be arranged perpendicular to the depth of the concave or convex piece 702. The joining edges 704 of the two pieces 702 may be configured to abut one another, to form the device 100. In some embodiments, an adhesive or epoxy may be used to couple the joining edges 704 of the two pieces 702 together. In other embodiments, the two pieces 702 may be coupled together by soldering along the joining edges 704. In some embodiments, a soldering metal may be one that has a color similar to the color of the oval pieces 702, so as to mitigate color differential between the pieces 702 and the solder line. For example, where the device comprises a copper color, the soldering component may have a similar copper color. In other embodiments, a silver or silver-based solder may be used. In still other embodiments, the two pieces 702 may be joined along the joining edges 704 by brazing or welding.

In some embodiments, the joining edges 704 may be configured to engage one another to help position the two halves during soldering or other joining operations. For example, as shown in FIG. 7, at least a portion of the joining edge 704 of each piece 702 may have a toothed or jagged surface. The teeth of the joining edge 704 on a first piece 502 may be configured to engage with the teeth of a joining edge on a second piece. The teeth may have any suitable spacing. In some embodiments, the toothed edge 704 may be created or formed when the ovals are cut from the copper-containing sheet or plate. For example, the ovals may be cute from a plate or sheet of material to have a jagged or toothed edge. In still other embodiments, one half of the device may have a joining edge with a lip or tab, and the opposing half of the device may have a recess or groove configured to receive the lip or tab when the two halves are arranged together along the joining edges to form the device.

In some embodiments, a device of the present disclosure may be buffed. For example, where the device comprises two pieces soldered, welded, brazed, or otherwise joined together, the device may be buffed in order to smooth the joint between the two pieces. Alternatively, where the device comprises a single cast piece of copper alloy, the device may be buffed after casting in order to generally smooth the surface and remove imperfections from the casting process.

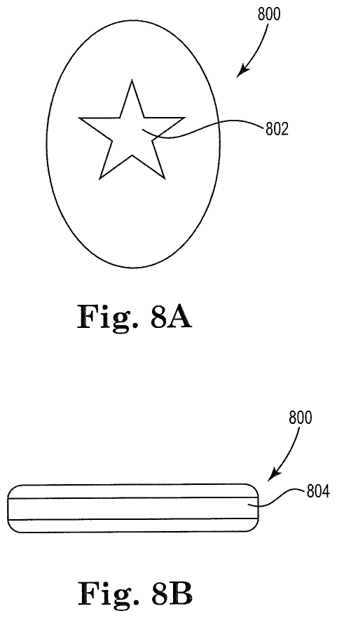

While in some embodiments, the device may be constructed entirely or mostly of a copper-containing compound or other antimicrobial material, in other embodiments, the device may have one or more inserts constructed of a copper-containing compound or other antimicrobial material. For example, a device of the present disclosure may be constructed primarily of plastic, glass, wood, or metals such as aluminum, steel, tin, silver, nickel, gold, or other metals. One or more copper-containing inserts having any suitable shape may be arranged on a surface of the device, such that the copper-containing inserts may make contact with a user's skin when the device is held or used. For example, FIG. 8A shows one embodiments of an antimicrobial device 800 of the present disclosure with an antimicrobial insert 802 embedded into or coupled to a surface of the antimicrobial device. The antimicrobial insert 802 may comprise copper, bronze, brass, and/or another antimicrobial material, as described above. The insert 802 may have any desirable shape and size on a surface of the device. For example, the insert may have a geometric shape, such as a star, as shown in FIG. 8A. Other geometric shapes are contemplated as well. In other embodiments, the insert 802 may have a shape of a letter, animal, or other known or whimsical object. Additionally or alternatively, in some embodiments, a perimeter or outer edge of a device may comprise copper or an antimicrobial material. As shown in FIG. 8B, for example, a perimeter surface 804 of the antimicrobial device 800 of the present disclosure may have an antimicrobial material. As another example, a perimeter of the smartphone case may be constructed of, or may have, a copper-containing compound or other antimicrobial material.

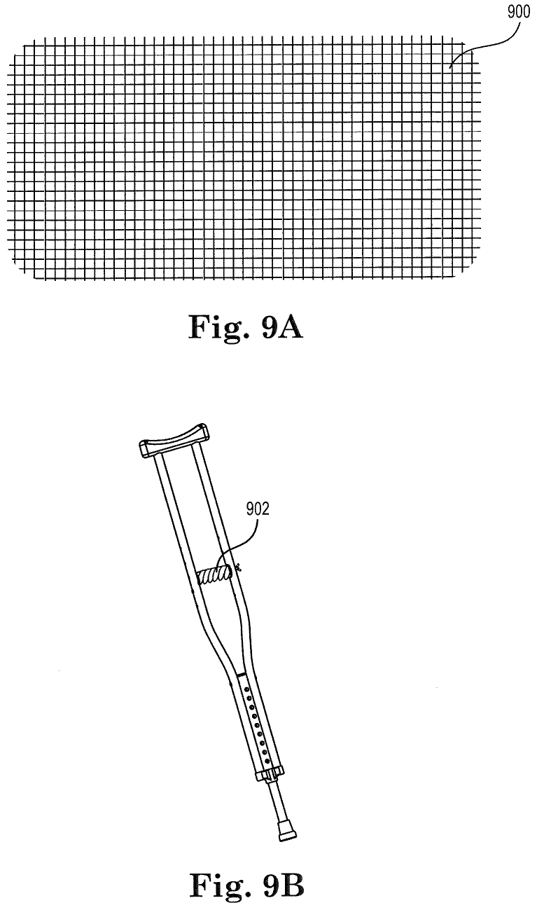

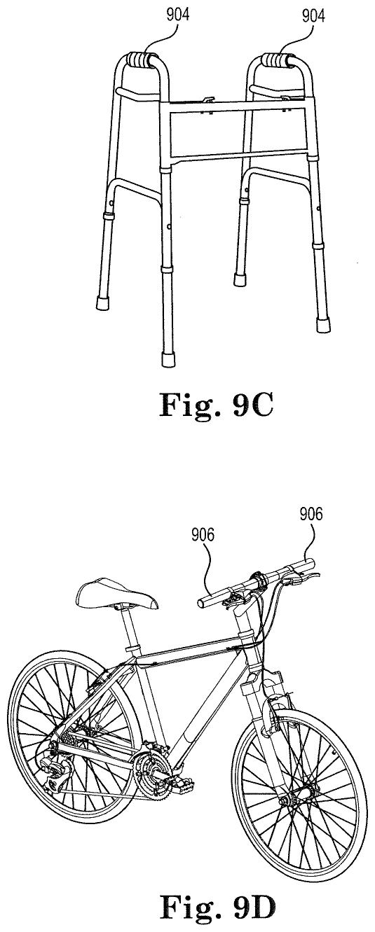

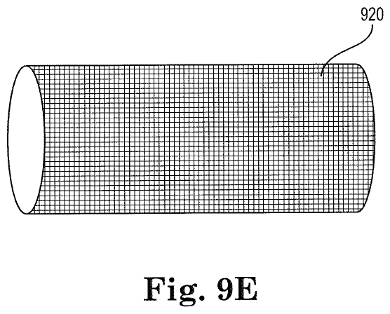

In another embodiment, an antimicrobial device of the present disclosure may be or include a fabric, mesh, or webbing comprising a copper-containing compound. As shown for example in FIG. 9A, copper-containing strands or wires may be woven together to form a copper-containing fabric or webbing 900. The fabric 900 may be generally stretchable, formable, or wrappable in at least one direction. The fabric 900 may have any suitable opening size formed between woven strands. For example, the openings between woven strands may range in size between approximately 0.1 inches and approximately 2 inches. In other embodiments, the openings between woven strands may have any other suitable size. Additionally, the fabric 900 may have any suitable mesh size, which may be a number of openings in a square inch of the fabric. For example, the fabric 900 may have a mesh size of between approximately 1 and approximately 1,000. The fabric 900 may be configured to be wrapped around or stretched over a surface that may come in frequent contact with a user's hands or skin. For example, the fabric 900 may be arranged about a crutch handle 902, as shown in FIG. 9B. In another embodiment, the fabric 900 may be wrapped about a handle 904 of a walker, as shown in FIG. 9C. In another embodiment, the fabric 900 may be wrapped about a bicycle handlebar 906, as shown in FIG. 9D. In some embodiments, a copper-containing fabric may include other materials or fibers. For example, copper-containing strands may be interwoven with copper or nylon strands or other materials. In at least one embodiments, a towel, such as a dish towel, may be provided with copper-containing strands interwoven with the fibers of the towel.

In some embodiments, the fabric 900 may be configured to adhere to a surface, such as a handle surface, using an adhesive or epoxy. For example, a liquid adhesive or epoxy may be applied to the crutch handle 902 prior to wrapping the fabric 900 over the handle. In other embodiments, the adhesive or epoxy may be arranged on one side of the fabric 900. For example, in some embodiments, the fabric 900 may have an adhesive side with a removable backing arranged over the adhesive, such that a user may remove the removable backing to adhere the fabric to a desired handle or other surface. In other embodiments, an adhesive tape may be used to couple the fabric 900 to a handle or other suitable surface. In other embodiments, the fabric 900 may be held in place over the handle using a length of wire tied over the fabric, for example. In still other embodiments, other coupling mechanisms may be used to couple the fabric 900 to a handle or other surface.

In some embodiments, the fabric 900 may be cut to have any suitable size and shape configured to fit the handle 902 or other suitable surface. For example, the fabric 900 may be cut to have a rectangular shape, with a width configured to fit across a length of a handle, and a length configured to wrap around a diameter of the handle. The fabric 900 may be wrapped around the handle 902 or other surface any suitable number of times, such that it may overlap itself in some embodiments. In other embodiments, the fabric 900 may be provided with a sheath shape, such that it may be stretched over a handle or other surface. For example, as shown in FIG. 9E, the fabric 900 may be arranged into an elongated sheath 920, open at each of two ends. The sheath 920 may be configured to be arranged over a handlebar, such as a handlebar on a crutch, cane, bicycle, or other suitable surface. In some embodiments, the sheath 920 may be stretchable, such that a user may stretch the sheath to fit over the handle or other surface. In this way, the sheath 920 may stay in place on the handle or other surface once it is stretched to fit the handle, due to compressive forces. The sheath may have any suitable diameter and length, and may be configured to fit a variety of handle types and/or sizes.

In use, a user may rub an antimicrobial device of the present disclosure on, over, and/or between the user's hands. For example, a user may rub the device on or between the user's hands for at least a minimum period of time, such as for at least 15 seconds, 30 seconds, 45 seconds, 60 second, 1.5 minutes, 2 minutes, or longer. In some cases, efficacy of the device may increase the longer it is rubbed. That is, the longer a user rubs the device, the more contaminants on the user's hands may be reduced or eliminated. However, even a short period of rubbing may reduce or substantially reduce contaminants on a user's skin. In some embodiments, devices of the present disclosure may help to reduce contaminants including, but not limited to, Enterobacter aeorgenes, Staphylococcus aureus, Pseudomonas aerugnosa, methicillin resistant Staphylococcus aureus, vancomycin resistant Staphylococcus aureus, Clostridium difficile, influenza A virus, adenovirus, fungi, and/or others.

It is to be appreciated that an antimicrobial device of the present disclosure may be used to reduce or minimize contaminants on a user's hands as needed and on the go. In this way, a device of the present disclosure may be used to decrease harmful or unwanted microbes on a user's hands without the need for soap, water, or other liquids. Thus, hand contaminants may be reduced on the go, or when soap and/or water may be inaccessible. The device may be easily carried, such as in a pocket or purse, and may thus allow a user to decrease or minimize contaminants in generally any public or private location. Devices of the present disclosure may be particularly useful where water, clean water, and/or soap are not readily accessible or available. Additionally, an antimicrobial device of the present disclosure may be used without the need for any cleanup and without creating any mess. Moreover, an antimicrobial device of the present disclosure may be used without drying a user's skin, unlike many antibacterial solutions. Devices of the present disclosure may also be used without producing any harmful effects to the user, and without creating any waste.

In addition, a handheld antimicrobial device of the present disclosure may provide additional benefits for the user. For example, the act of rubbing the device between a user's hands may be soothing or therapeutic for a user. In particular, the device's smooth surfaces may be soothing for a user. Moreover, as described above, the device may provide massaging benefits with protuberances or other massaging components.

As described below, independent laboratories were commissioned to conduct tests demonstrating the antimicrobial benefits of devices of the present disclosure.

### Test 1: Antimicrobial Device Rubbed on Imitation Skin for 3 Minutes, Minimal Pressure

Using an ex-vivo skin model, this test evaluated the effectiveness of a device of the present disclosure in reducing single selected microorganisms. A handheld antimicrobial device of the present disclosure was constructed using a sheet of 0.050-inch thick copper. The copper was Uniform Number System (UNS)C-220 copper, having approximately 90% copper and approximately 10% zinc. The antimicrobial device was tested for its efficacy in minimizing two contaminants: Escherichia coli (ATCG 112291) and Staphylococcus aureus (ATCC 6538). The growth medium used for the test organisms was tryptic soy agar with 5% sheep blood (BAP). VITRO-SKIN® was used as a testing substrate to mimic surface properties of human skin. VITRO-SKIN® contains both optimized protein and lipid components and is designed to have topography, pH, critical surface tension and ionic strength similar to human skin.

Four 1.5-inch by 1.5-inch VITRO-SKIN® carriers were obtained. Two of the four carriers were treated with Escherichia coli, and two of the four carriers were treated with Staphylococcus aureus. In particular, on each of two carriers, a 1-inch by 1-inch film of Escherichia coli was applied and allowed to dry. Additionally, on each of two carriers, a 1-inch by 1-inch film of Staphylococcus aureus was applied to a second carrier, and allowed to dry. Population controls were performed to assess the number of colony forming units (CFU) on each carrier. The table below shows the population control results. As shown below, the geometric mean CFU/carrier for the two Escherichia coli carriers was 2.75×10⁵, and the geometric mean CFU/carrier for the two Staphylococcus aureus carriers was 1.12×10⁴.

TABLE 1
Population Control Results

| | Carrier | | Log₁₀ of | Average | Geometric Mean |
|---|---|---|---|---|---|
| Test Organism | # | CFU/Carrier | CFU/Carrier | Log₁₀ | (CFU/Carrier) |
| Escherichia coli | 1 | 2.82 × 10⁵ | 5.45 | 5.44 | 2.75 × 10⁵ |
| (ATCC 11229) | 2 | 2.70 × 10⁵ | 5.43 | | |
| Staphylococcus | 1 | 8.0 × 10³ | 3.90 | 4.05 | 1.12 × 10⁴ |
| aureus | 2 | 1.6 × 10⁴ | 4.20 | | |
| (ATCC 6538) | | | | | |

Each of the four carriers, having dried test organisms thereon, was treated by rubbing the C-220 copper test substance over the inoculated surface for 3 minutes at ambient conditions. Minimal pressure was used for the rubbing. After exposure, each carrier was neutralized with letheen broth (20 mL). After neutralization, the carriers were assayed for survivors by dilution and plating at various dilutions. Tables 2 and 3, below, show the number of colonies counted in each plated dilution. Percent and Logic) reductions were determined for the test based on the test population control results.

TABLE 2
Test Results Against Escherichia coli (ATCC 11229)

| | |
|---|---|
| Dilution | Survivors |

| (Volume Plated) | Replicate #1 | Replicate #2 |
|---|---|---|
| 10⁰ (1.00 mL) | T, T | T, T |
| 10⁰ (0.100 mL) | 132, 142 | 298, 212 |
| 10⁻¹ (0.100 mL) | 13, 21 | 33, 25 |
| 10⁻² (0.100 mL) | 4, 1 | 3, 4 |
| CFU/Carrier | 2.74 × 10⁴ | 5.8 × 10⁴ |
| Log₁₀ CFU/Carrier | 4.44 | 4.76 |

| | |
|---|---|
| Average Log₁₀ | 4.60 |
| Geometric Mean | 3.98 × 10⁴ |
| Log₁₀ Reduction = | 0.84 |
| Percent Reduction = | 85.5% |

CFU = Colony Forming Units
T = Too Numerous To Count (>300 colonies)

TABLE 3
Test Results Against Staphylococcus aureus (ATCC 6538)

| | |
|---|---|
| Dilution | Survivors |

| (Volume Plated) | Replicate #1 | Replicate #2 |
|---|---|---|
| 10⁰ (1.00 mL) | 110, 115 | 89, 96 |
| 10⁰ (0.100 mL) | 7, 9 | 8, 19 |
| 10⁻¹ (0.100 mL) | 0, 1 | 0, 0 |
| 10⁻² (0.100 mL) | 0, 0 | 0, 0 |
| CFU/Carrier | 2.26 × 10³ | 1.9 × 10³ |
| Log₁₀ CFU/Carrier | 3.35 | 3.28 |

| | |
|---|---|
| Average Log₁₀ | 3.32 |
| Geometric Mean | 2.09 × 10³ |
| Log₁₀ Reduction = | 0.73 |
| Percent Reduction = | 81.3% |

CFU = Colony Forming Units

As shown in the above tables, the exposure to the copper sample resulted in approximately an 85.5% reduction of Escherichia coli and approximately an 81.3% reduction of Staphylococcus aureus.

Test 2: Antimicrobial Device Rubbed on Imitation Skin for 1 Minute, Light to Medium Pressure

Using an ex-vivo skin model, this test evaluated the effectiveness of a device of the present disclosure in reducing single selected microorganisms. A handheld antimicrobial device of the present disclosure was constructed using a sheet of 0.050-inch thick copper. The copper was Uniform Number System (UNS)C-220 copper, having approximately 90% copper and approximately 10% zinc. The antimicrobial device was tested for its efficacy in minimizing Staphylococcus aureus (ATCC 6538). The growth medium used for the test organism was tryptic soy agar with 5% sheep blood (BAP). VITRO-SKIN® was used as an testing substrate to mimic surface properties of human skin. VITRO-SKIN® contains both optimized protein and lipid components and is designed to have topography, pH, critical surface tension and ionic strength similar to human skin.

Two 1.5-inch by 1.5-inch VITRO-SKIN® carriers were obtained. On each of the two carriers, a 1-inch by 1-inch film of Staphylococcus aureus was applied and allowed to dry. Population controls were performed to assess the number of colony forming units (CFU) on each carrier. The table below shows the population control results. As shown below, the geometric mean CFU/carrier for the two Staphylococcus aureus carriers was 2.40×10⁵.

TABLE 4
Population Control Results

| | Carrier | | Log₁₀ of | Average | Geometric Mean |
|---|---|---|---|---|---|
| Test Organism | # | CFU/Carrier | CFU/Carrier | Log₁₀ | (CFU/Carrier) |
| Staphylococcus | 1 | 2.36 × 10⁵ | 5.37 | 5.38 | 2.40 × 10⁵ |
| aureus | 2 | 2.44 × 10⁵ | 5.39 | | |
| (ATCC 6538) | | | | | |

CFU = Colony Forming Unit

Each of the two carriers, having dried test organisms thereon, was treated by rubbing the C-220 copper test substance over the inoculated surface for 1 minute at ambient conditions. Light to medium pressure was used for the rubbing. The rubbing was intended to replicate a user's use of the device, in rubbing the device on the user's hands. After exposure, each carrier was neutralized with letheen broth (20 mL). After neutralization, the carriers were assayed for survivors by dilution and plating at various dilutions. Table 5, below, shows the number of colonies counted in each plated dilution. Percent and Log₁₀ reductions were determined for the test based on the test population control results.

TABLE 5
Test Results Against Staphylococcus aureus (ATCC 6538)

| | |
|---|---|
| Dilution | Survivors |

| (Volume Plated) | Replicate #1 | Replicate #2 |
|---|---|---|
| 10⁰ (1.00 mL) | T, T | T, T |
| 10⁰ (0.100 mL) | 86, 83 | 53, 63 |
| 10⁻¹ (0.100 mL) | 12, 12 | 7, 5 |
| 10⁻² (0.100 mL) | 1, 0 | 2, 2 |
| CFU/Carrier | 1.7 × 10⁴ | 1.2 × 10⁴ |
| Log₁₀ CFU/Carrier | 4.23 | 4.08 |

| | |
|---|---|
| Average Log₁₀ | 4.16 |
| Geometric Mean | 1.45 × 10⁴ |
| Log₁₀ Reduction = | 1.22 |
| Percent Reduction = | 94.0% |

CFU = Colony Forming Units
T = Too Numerous to Count (>300)

As shown in the above tables, the exposure to the copper-containing device resulted in a 94% reduction of Staphylococcus aureus.

Test 3: Zone of Inhibition Test

The third test involved zone of inhibition (or Kriby-Bauer) testing methodology. To prepare the inoculum, a stock culture of Staph aureus ATCC 25923 and Salmonella ATCC 14028 were streaked onto the following selective agars and incubated at 35 degrees Celsius for 24 hours.

TABLE 6
Inoculum Prep

| Agar | Uninoculated | Inoculated |
|---|---|---|
| Staph - Baird Parker | Agar is very light | Staph will from colonies |
| | yellow in color | are black |
| Salmonella - XLD | Agar is red | Salmonella will form |
| | | black colonies |

The surface of each 24-hour culture was flushed with approximately 5-10 mls of sterile phosphate buffer and scraped with a sterile hockey stick to remove the organisms. The rinsed buffer was collected into a 100 ml buffer rinse solution. One ml of inoculum was added to 500 ml of agars listed.

Pure copper chips were cut to have a 0.375-inch square shape with a thickness of approximately 0.04 inches. As further described below, a copper chip was placed over each of the Staphylococcus aureus seeded agar and the Salmonella seeded agar. Additionally, a copper chip was overlaid with each of the Staphylococcus aureus seeded agar and the Salmonella seeded agar.

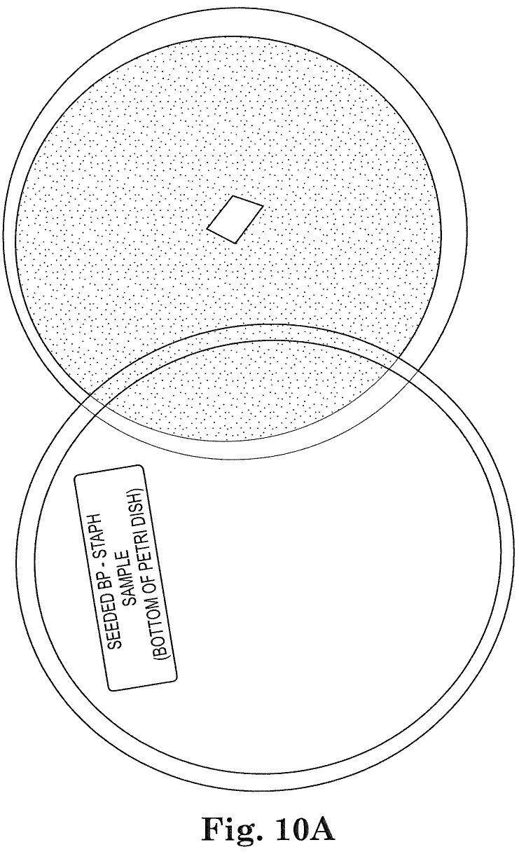

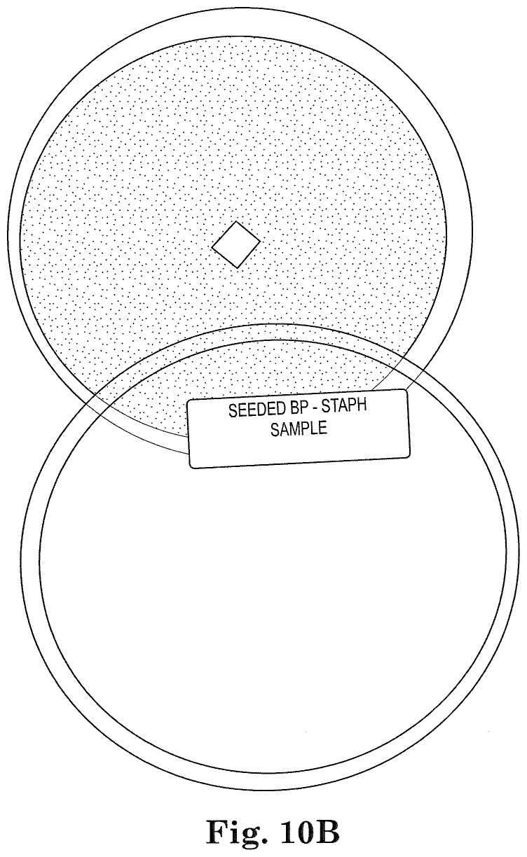

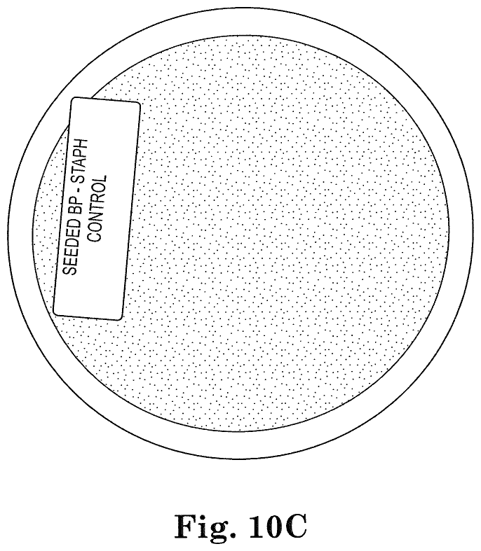

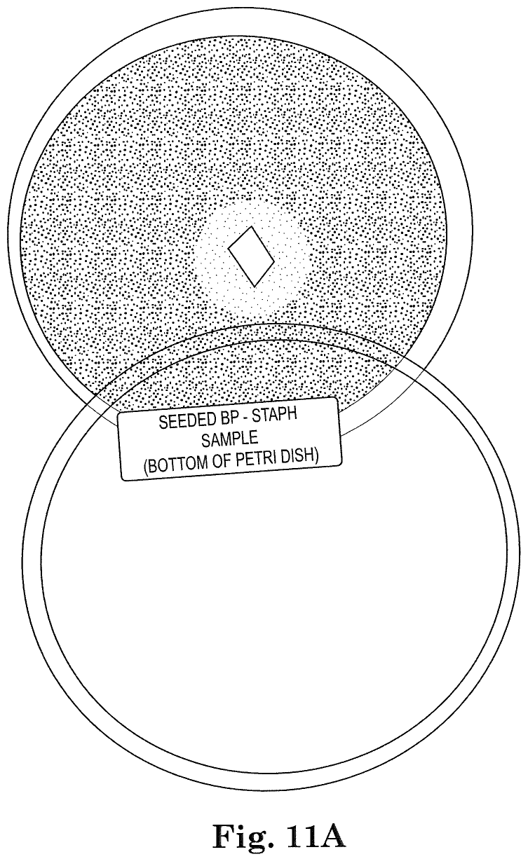

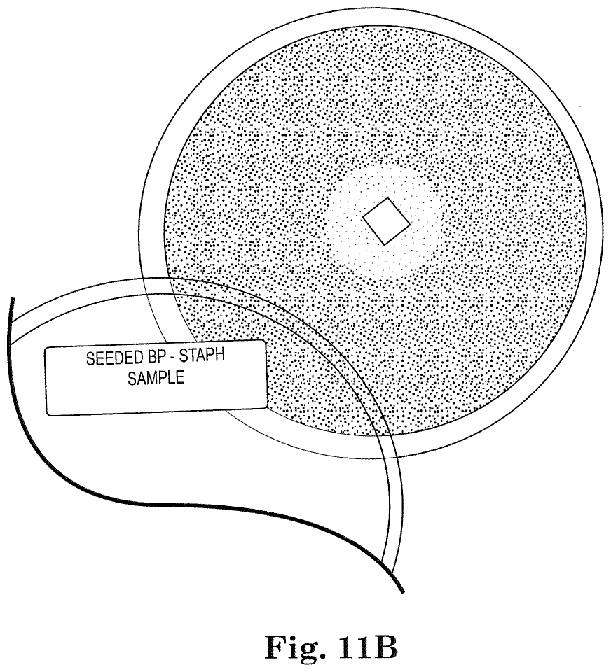

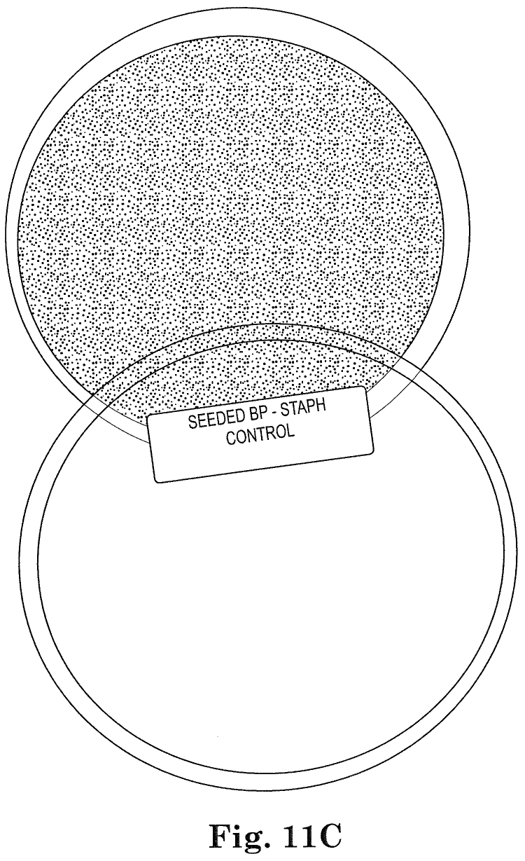

In a first petri dish, a copper chip was placed in an agar plate and overlaid with the Staphylococcus aureus seeded agar. FIG. 10A shows the first dish at time 0. In a second petri dish, a copper chip was placed over the Staphylococcus aureus seeded agar. FIG. 10B shows the second dish at time 0. A third petri dish provide a control, in which the Staphylococcus aureus seeded agar was placed. FIG. 10C shows the third petri dish at time 0. The three dishes were permitted to incubate for 18 hours and 35 degrees Celsius. FIGS. 11A, 11B, and 11C show the first, second and third (control) dishes, respectively, after the 18 hour incubation. As may be appreciated from FIGS. 11A and 11B, a visible zone of inhibition was formed around the copper chip in both the first a second dishes. That is, whether the chip was placed on the bottom of the dish and overlaid with the seeded agar, or was placed over the agar, a visible zone of inhibition formed.

Additionally, in a fourth petri dish, a copper chip was placed in an agar plate and overlaid with the Staphylococcus aureus seeded agar. In a fifth petri dish, a copper chip was placed over the Staphylococcus aureus seeded agar. A sixth petri dish provide a control, in which the Staphylococcus aureus seeded agar was placed. The three dishes were permitted to incubate for 18 hours and 35 degrees Celsius. After the incubation period, a visible zone of inhibition was formed around the copper chip in both the first a second dishes. That is, whether the chip was placed on the bottom of the dish and overlaid with the seeded agar, or was placed over the agar, a visible zone of inhibition formed.

The results of the zone of inhibition test are summarized in the table below. As may be appreciated from these results, the copper chip exhibited antimicrobial properties against both Salmonella and Staphylococcus aureus.

TABLE 7
Zone of Inhibition Test Results Against Staph and Salmonella
Zone of Inhibition

| | Salmonella | Staph |
|---|---|---|
| Copper Chip Placed on | Zone of Inhibition | Zone of Inhibition |
| Seeded Agar Surface | Present | Present |
| Copper Chip Placed on | Zone of Inhibition | Zone of Inhibition |
| Seeded Agar Bottom | Present | Present |

As used herein, the terms “substantially” or “generally” refer to the complete or nearly complete extent or degree of an action, characteristic, property, state, structure, item, or result. For example, an object that is “substantially” or “generally” enclosed would mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

1.-20. (canceled)

21. A method of reducing bacteria on a user's hands, the method comprising:
rubbing a device on or between the user's hands for at least 15 seconds to reduce bacteria on the user's hands, the device comprising:
a disk shaped device having a top surface, a bottom surface, and a side surface connecting the top surface to the bottom surface, wherein the top, bottom and side surfaces comprise antimicrobial surfaces comprising at least 60% copper, wherein all devices surfaces are smooth and all device edges are rounded;
wherein the device is sized to fit within a palm of the user's hand.

22. The method of claim 21 wherein the device has a length of between approximately 2 inches and 5 inches, a width between approximately 1 inches and 4 inches, and a depth between approximately 0.25 inches and 1 inch.

23. The method of claim 21 wherein rubbing the device on or between the user's hands comprises rubbing the device for at least 30 seconds.

24. The method of claim 21 wherein rubbing the device on or between the user's hands comprises rubbing the device for at least 60 seconds.

25. The method of claim 21 wherein the top, bottom and side surfaces comprise antimicrobial surfaces comprising at least 70% copper.

26. The method of claim 21 wherein the top, bottom and side surfaces comprise antimicrobial surfaces comprising at least 80% copper.

27. The method of claim 21 wherein the top, bottom and side surfaces comprise antimicrobial surfaces comprising at least 90% copper.

28. The method of claim 21 wherein the top, bottom and side surfaces further comprise at least one of tin, nickel, aluminum, and zinc.

29. The method of claim 21 wherein the device is circular or oval.

30. The method of claim 29 wherein the device is convex.

31. A method of reducing bacteria on a user's hands, the method comprising:
carrying a device in a mobile and accessible location, the device comprising:
a disk shaped device having a top surface, a bottom surface, and a side surface connecting the top surface to the bottom surface, wherein the top, bottom and side surfaces comprise antimicrobial surfaces comprising at least 60% copper, wherein all devices surfaces are smooth and all device edges are rounded;
wherein the device sized to fit within a palm of the user's hand; and
rubbing the device on or between the user's hands as needed on the go for at least 30 seconds.

32. The method of claim 31 wherein the mobile and accessible location comprises a purse or backpack.

33. The method of claim 31 wherein the mobile and accessible location comprises a pocket.

34. The method of claim 31 wherein rubbing the device on or between the user's hands comprises rubbing the device for at least 60 seconds.

35. The method of claim 31 wherein the top, bottom and side surfaces comprise antimicrobial surfaces comprising at least 70% copper.

36. The method of claim 31 wherein the top, bottom and side surfaces comprise antimicrobial surfaces comprising at least 80% copper.

37. The method of claim 31 wherein the device is circular or oval and convex.

38. A method of reducing bacteria on a user's hands, the method comprising:
washing a device prior to first use, the device comprising:
a disk shaped device having a top surface, a bottom surface, and a side surface connecting the top surface to the bottom surface, wherein the top, bottom and side surfaces comprise antimicrobial surfaces comprising at least 60% copper, wherein all devices surfaces are smooth and all device edges are rounded;
the device having length of between approximately 2 inches and 5 inches, a width between approximately 1 inches and 4 inches, and a depth between approximately 0.25 inches and 1 inch; and
after washing the device, rubbing a device on or between the user's hands to reduce bacteria on the user's hands.

39. The method of claim 38 wherein washing the device comprises washing the device in hydrogen peroxide or chlorine bleach.

40. The method of claim 38 wherein washing the device comprises washing the device to remove an initial coating or oil.
